(19) United States
(12) Patent Application Publication
(10) Pub. No.: US 2012/0298528 A1
(43) Pub. Date: Nov. 29, 2012

(54) BIOSENSOR AND ANALYSIS METHOD USING SAME

(76) Inventors: Naomi ASANO, Osaka (JP); Yuichiro Shimizu, Osaka (JP)

(21) Appl. No.: 13/419,456

(22) Filed: Mar. 14, 2012

(30) Foreign Application Priority Data
May 24, 2011 (JP) 2011-116211

(51) Int. Cl. G01N 27/26; G01N 33/50

(52) U.S. Cl. 205/775; 204/403.01; 204/403.14

(57) ABSTRACT

The present invention provides a biosensor including a working electrode or working electrodes on which a reaction material or a bonding material is immobilized, where the reaction material is reactive with a target material so as to produce a product, and the bonding material is bondable with a target material; a counter electrode; and a reaction section for holding a sample liquid containing the target material, the working electrode and the counter electrode being provided on a bottom surface of the reaction section, and the working electrode occupying the bottom surface of the reaction section by a ratio of 0.7 or greater.

This Nonprovisional application claims priority under 35 U.S.C. §119(a) on Patent Application No. 2011-116211 filed in Japan on May 24, 2011, the entire contents of which are hereby incorporated by reference.

### TECHNICAL FIELD

The present invention relates to a biosensor suitable for use in analyzing biological objects, environments, medical objects, and foods, etc., and an analysis method using the same.

### BACKGROUND ART

Electrochemical measuring methods based on electrochemical reactions in solutions are widely employed in analysis for biological objects, environments, medical objects, and foods, etc. For example, there are electrochemical measuring methods using enzymic electrodes for measuring materials (sugar, neutral lipid, etc.) in biological samples.

Moreover, for analysis of minute amounts of materials (proteins, hormones, etc.) in biological samples, immunoanalytical methods of electrochemical detecting types are widely used. Electrodes used in the electrochemical measurement in these analysis have such configuration that predetermined electrodes (working electrodes, counter electrodes, reference electrodes, etc.) made form an electrically conductive material (s) and that a reacting material (enzyme, antibody, peptide, or the like) is immobilized on the electrodes. With this configuration, a target material is electrochemically detected based on an ELISA reaction or an enzyme-substrate reaction occurring on or in the vicinity of the electrodes.

The following patent literatures disclose invention using the electrochemical measuring methods.

Patent Literature 1 discloses a biosensor including a working electrode (measuring electrode) and a counter electrode provided on an insulating substrate, and a reaction layer being in touch with these electrodes and containing an enzyme or the like.

Patent Literature 2 discloses a biosensor including a working electrode (measuring electrode) and a counter electrode provided on an insulating substrate, a polymer layer on or in the vicinity of these electrodes, and a filter paper layer on the polymer layer, the filter paper layer supporting a neutral lipolytic enzyme.

Patent Literature 3 discloses a flat plate-shaped electrode serving as a working electrode, a counter electrode, and a reference electrode, which are formed by patterning an electrically conductive material on an insulating substrate, and also discloses an electrochemical detecting sensor in which an enzyme is immobilized on the working electrode formed in a flat plate-shaped electrode.

Patent Literature 4 discloses an immunoassay electrochemical sensor in which an antibody is covalently immobilized on a metal electrode provided on an insulating substrate.

### CITATION LIST

### Patent Literatures

Patent Literature 1
Japanese Patent Application Publication, Tokukai, No. 2001-174432 A (Publication Date: Jun. 29, 2001)

Patent Literature 2
Japanese Patent Application Publication, Tokukai, No. 2009-139114 A (Publication Date: Jun. 25, 2009)

Patent Literature 3
Japanese Patent Application Publication, Tokukai, No. 2007-278981 A (Publication Date: Oct. 25, 2007)

Patent Literature 4
Japanese Patent Application Publication, Tokukai, No. 2009-244013 A (Publication Date: Oct. 22, 2009)

### SUMMARY OF INVENTION

### Technical Problem

It is desirable to provide a biosensor capable of detecting more accurately. Especially, it is desirable to provide a biosensor capable of detecting accurately even in a short period.

The present invention was accomplished in view of the problems. An object of the present invention is to provide a biosensor capable of detecting accurately even in a short period, and an analysis method using the same.

### Solution to Problem

The inventors of the present invention made diligent studies on this object. As a result, the inventors of the present invention found via simulation that accuracy of electrochemical analysis is influenced by an area ratio between an area of the working electrode and a bottom area which is in touch with a liquid to be subjected to the electrochemical measurement. The present invention is accomplished based on this finding.

The simulation also demonstrated that a difference between a theoretical initial reaction rate and an actual initial reaction rate becomes smaller when the area ratio (working electrode-bottom surface area ratio) of the area of the working electrode to the bottom area that is in touch with the liquid to be subjected to the electrochemical measurement is larger. Moreover, the simulation further demonstrated that the theoretical initial reaction rate and the actual initial reaction rate become substantially equal to each other when the working electrode-bottom surface area ratio is 0.7 or greater. Note that the simulation will be described later in detail.

In order to attain the object, a biosensor according to the present invention is a biosensor including a working electrode or working electrodes on which a reaction material or a bonding material is immobilized, where the reaction material is reactive with a target material so as to produce a product, and the bonding material is bondable with a target material; a counter electrode; and a reaction section for holding a sample liquid containing the target material, the working electrode and the counter electrode being provided on a bottom surface of the reaction section, and the working electrode occupying the bottom surface of the reaction section by a ratio of 0.7 or greater.

Compared with the conventional biosensor, this configuration makes it possible to reduce a difference between an actual initial reaction rate and a calculated initial reaction rate in an reaction initial stage of a reaction for producing the product, the calculated initial reaction rate being obtained from a gradient of a straight line connecting an origin and a product amount at a given time. Therefore, this configuration makes it possible to perform accurate detection with a short reaction time without requiring to wait for the reaction to saturate.

This is explained herein for further details. The conventional biosensors (Patent Literatures 1 to 4) is configured such that the area ratio of the working electrode (measuring electrode) to an area to be in touch with the sample liquid or measuring-target liquid on a substrate is small. This is because these conventional biosensors are so configured that the counter electrode for flowing the current caused by the working electrode has an area ratio substantially equal to or greater than that of the working electrode in order to avoid difficulty in flowing the current through the counter electrode. Thus, the conventional biosensors are so configured that the working electrode occupies, by an area ratio of 0.5 or less, the area to be in touch with the liquid.

Here, in general, the product produced on the working electrode gradually move away from the working electrode by diffusion. The electrochemical detection is capable of detecting only the product present in the vicinity of the working electrode. If the area ratio of the function electrode to the bottom area of the reaction section is 0.5 as in the conventional biosensors, a portion not the working electrode is large in the bottom area of the reaction section. This follows that an amount of the product moving out of detectable range due to the diffusion is large. Consequently, the reaction time and the product amount (product amount on the working electrode) has low linearity in the reaction initial stage.

On the other hand, in the biosensor in which the area ratio of the working electrode to the bottom surface of the reaction section as described above is 0.7 or greater, the portion not the working electrode is small in the bottom area of the reaction section. This follows that an amount of the product moving out of detectable range due to the diffusion is small. Consequently, the reaction time and the product amount (product amount on the working electrode) has high linearity even in the reaction initial stage, that is, the reaction time and the product amount has a more linear relationship therebetween. Because of this, this configuration makes it possible to reduce a difference between an actual initial reaction rate and a calculated initial reaction rate in an reaction initial stage of a reaction for producing the product, the calculated initial reaction rate being obtained from a gradient of a straight line connecting an origin and a product amount at a given time, whereby this configuration makes it possible to perform accurate detection even with a short reaction time.

In order to attain the object, an analysis method according to the present invention is an analysis method using the aforementioned biosensor, including: introducing, to the reaction section, the sample liquid containing the target material; and measuring an ampere value of a current caused by voltage application between the working electrode and the counter electrode, the ampere value being varied according to an amount of the target material reacted or bonded.

With this configuration, the step of introducing introduces the sample liquid containing the target material to the reaction section. This causes reaction between the reaction material immobilized on the working electrode and the target material or bonding between the bonding material on the working electrode and the target material. By applying a voltage between the working electrode and the counter electrode, an ampere value being varied according to an amount of the target material reacted or bonded can be obtained. The configuration of the present invention provides a high linearity (more liner relationship) between the reaction time and the product amount of the product in the reaction initial stage. Because of this, this configuration makes it possible to reduce a difference between an actual initial reaction rate and a calculated initial reaction rate in an reaction initial stage of a reaction for producing the product, the calculated initial reaction rate being obtained from a gradient of a straight line connecting an origin and a product amount at a given time with in a short reaction time, whereby this configuration makes it possible to perform accurate detection even with a short reaction time.

### Advantageous Effects of Invention

A biosensor according to the present invention is a biosensor including a working electrode or working electrodes on which a reaction material or a bonding material is immobilized, where the reaction material is reactive with a target material so as to produce a product, and the bonding material is bondable with a target material; a counter electrode; and a reaction section for holding a sample liquid containing the target material, the working electrode and the counter electrode being provided on a bottom surface of the reaction section, and the working electrode occupying the bottom surface of the reaction section by a ratio of 0.7 or greater. This configuration makes it possible to perform accurate detection with a short reaction time without requiring to wait for the reaction to saturate.

### BRIEF DESCRIPTION OF DRAWINGS

FIG. 1 is a top view schematically illustrating a configuration of a biosensor 100 according to Embodiment 1.

FIG. 2 is a top view schematically illustrating a configuration of a biosensor 200 according to Embodiment 2.

FIG. 3 is a top view schematically illustrating a configuration of a biosensor 300 according to Embodiment 3.

FIG. 4 is a top view schematically illustrating a configuration of a biosensor 400 according to Embodiment 4.

FIG. 5 is a top view schematically illustrating a configuration of a biosensor 500 according to Embodiment 5.

FIG. 6 is a top view schematically illustrating a configuration of a biosensor 500′ according to a modification of Embodiment 5.

FIG. 7 is a top view schematically illustrating a configuration of a biosensor 600 according to Embodiment 6.

FIG. 8 is a perspective view schematically illustrating the configuration of the biosensor 600 according to Embodiment 6.

FIG. 9 is a view schematically illustrating a top view and a cross sectional view of a configuration of a biosensor 700 according to Embodiment 7.

FIG. 10 is a perspective view schematically illustrating the configuration of the biosensor 700 according to Embodiment 7.

FIG. 11 is a top view schematically illustrating a configuration of a biosensor used in Examples.

FIG. 12 is a view plotting an amount of a product against time in a general enzyme-substrate reaction.

FIG. 13 is a view illustrating relationship between an amount of a product formed on a working electrode and time in a conventional biosensor.

FIG. 14 is a view illustrating relationship between an amount of a product formed on a working electrode and time in a biosensor according to the present invention.

FIG. 15 is a view in which R² is plotted against area ratios of the working electrode, where R is a linear correlation function of an approximate straight line in a reaction initial stage in a curve indicating a relationship between a product amount on a working electrode and time.

### DESCRIPTION OF EMBODIMENTS

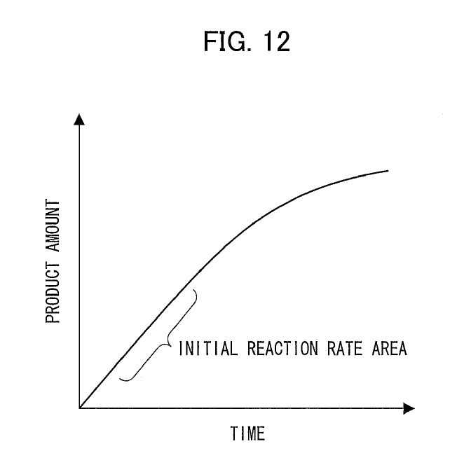

FIG. 12 is a graph plotting an amount of a product against time in an enzyme-substrate reaction in an ELISA method or an enzymic method. The enzyme-substrate reaction taken place in the ELISA method or the enzymic method shows a linear relationship between time and a total amount of the product in the reaction initial stage in general. The linear relationship has a gradient equal to an initial reaction rate of the enzymic-substrate reaction. An area corresponding to an initial stage of the reaction is referred to as an initial reaction rate area.

In case where the enzyme is abundant with respect to the substrate, the initial reaction rate is proportional to a substrate concentration. In case where the target material is a substrate (for example, in case of a glucose sensor targeting glucose as its target material), the proportionality of the initial reaction rate and the substrate concentration allows to determine the substrate concentration, that is, the target concentration by finding the initial reaction rate.

Moreover, in case where the substrate is abundant with respect to the enzyme, the initial reaction rate is proportional to an enzyme concentration. In case where the target material is detected by using a material (such as antibody) being bondable with the target material (in case of immunoassay if an antibody is used), the proportionality of the initial reaction rate and the enzyme concentration allows to determine the enzyme concentration by finding the initial reaction rate. The enzyme concentration is a concentration of an enzyme-labeled antibody bonded with the target material. That is, the enzyme concentration can be determined as an indicator of the target material concentration of the target material bonded to the enzyme-labeled antibody.

As described above, the initial reaction rate of the enzyme-substrate reaction is a very important factor to determine the substrate concentration and the enzyme concentration in the measuring system.

Especially, in case where an immune reaction, enzyme-substrate reaction, or the like is detected electrochemically by using the biosensor as described above, it is important that the reaction in the biosensor take place with a linearity between a detected ampere value (being proportional to the product amount) and time in the reaction initial stage. However, the electrochemical method is capable of quantitatively detecting only such a product that is present in a limited region on the working electrode, but is not capable of a total amount of the product. Thus, it is not easy to carry out the reaction with linearity in the biosensor. This is because the electrochemical method cannot measure the total amount of a product since the product produced on the working electrode is moved out of the region due to mass transfer caused by diffusion.

The detected ampere value is proportional to the product amount. Therefore, the detected ampere value is referred to as the product amount hereinafter.

In case where a reaction takes place with a linearity between an amount (product amount) of a product produced from the reaction and time (reaction time) elapsed in the reaction, an initial reaction rate of the reaction can be obtained as a value obtained by dividing the product amount at a given reaction time by the reaction time, that is a gradient of a straight line connecting an origin and the product amount at the reaction time on the graph plotting the product amount against the reaction time. However, in the case of the general (conventional) biosensor and electrochemical detecting sensor, the linearity between the product amount and the time is so low that the gradient of the straight line thus obtained does not represent the actual initial reaction rate. Therefore, the product amount calculated out based on the initial reaction rate obtained from the straight line (approximate straight line) has a large error from the actual product amount. Thus, in order to carry out accurate detection, the electrochemical measurement should be performed after the reaction is saturated to become stable. That is, in order to accurately perform the detection, it has been conventionally required to performed the detection for a certain length of time. If the detection is performed in a short time, the detection becomes inaccurate conventionally.

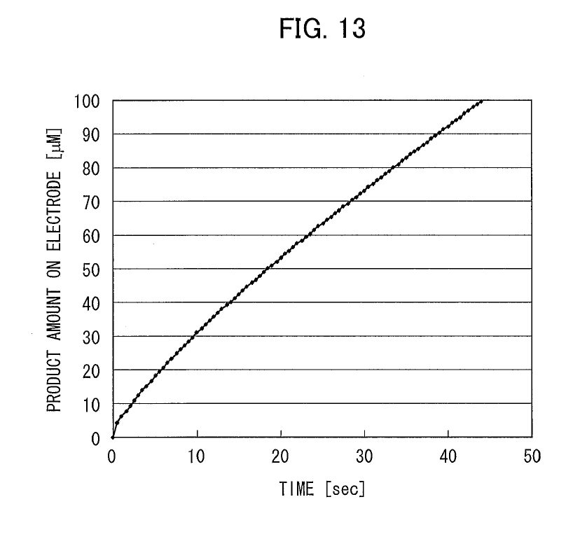

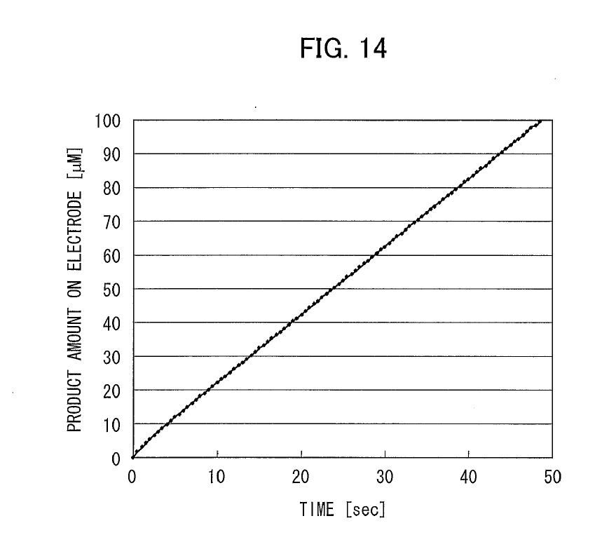

FIG. 13 shows results of time and a product amount in a detectable region on the working electrode in a conventional biosensor in which the ratio of the area of the working electrode to the bottom area which is in touch with a liquid to be subjected to the electrochemical measurement is 0.5. FIG. 14 shows results of time and a product amount in a detectable region on the working electrode in a biosensor of the present invention in which the ratio of the area of the working electrode to the bottom area which is in touch with a liquid to be subjected to the electrochemical measurement is 0.7. Both FIGS. 13 and 14 show the results in the reaction initial stage, which is up to about several tens sec from the start of the reaction.

Here, the linearity is evaluated, based on R², as to how linear it is. R² is a square value of a linear correlation function of the approximate straight line. As R² approximates to 1, the linearity between the time and the product amount becomes more linear. In the conventional biosensor, R² of the approximate straight line was 0.9928 in the time period from 1 to 10 sec in the graph of FIG. 13. On the other hand, in the biosensor of the present invention, R² of the approximate straight line was 0.9990 in the time period from 1 to 10 sec in the graph of FIG. 14. This shows that, in the electrochemical detecting biosensor with the area ratio of 0.5, the linearity between the time and product amount is not so high and the initial reaction rate obtained from the gradient of the line between the origin and a point at a given time does not faithfully represent the actual initial reaction rate. On the other hand, in the biosensor with the area ratio of 0.7, the linearity between the time and the product amount is high and the approximate straight line more faithfully represents the actual product amount at a given time.

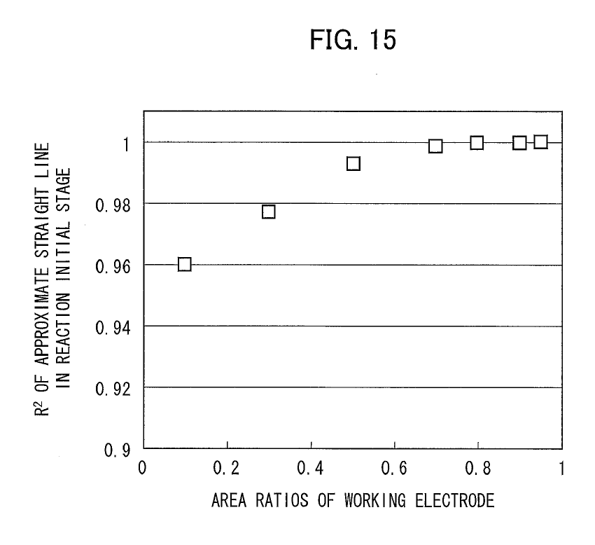

Further, FIG. 15 is a graph in which R² (where R is the liner correlation function of the approximate straight line) in the reaction initial stage (0 to 10 sec) is plotted against area ratios of the area of the working electrode to the bottom area which is in touch with a liquid to be subjected to the electrochemical measurement. FIG. 15 shows that the working electrode with a greater area ratio had R² more approximate to 1. With the area ratios of the working electrode in a range of not less than 0.7 but less than 1, R² is 0.999 or greater, that is, substantially 1. On the other hand, as the area ratio becomes smaller below 0.7, R² becomes rapidly smaller. With the area ratio of 0.1, R² is unfavorably reduced to 0.96. This concludes that the error between the rate obtained from the gradient of the straight line of the reaction and the actual initial reaction rate is small in a biosensor with a working electrode having an area ratio of 0.7 or greater when the biosensor perform the detection with a short reaction time with a smaller. It can be said that the rate obtained from the gradient of the straight line of the reaction and the actual initial reaction rate are substantially equal with each other in the biosensor with a working electrode having an area ratio of 0.7 or greater. That is, by giving the working electrode a greater area ratio of the area of the working electrode to the bottom area which is in touch with a liquid to be subjected to the electrochemical measurement, it becomes possible to more accurately detect the concentration of the target material even with a short reaction time.

### Embodiment 1

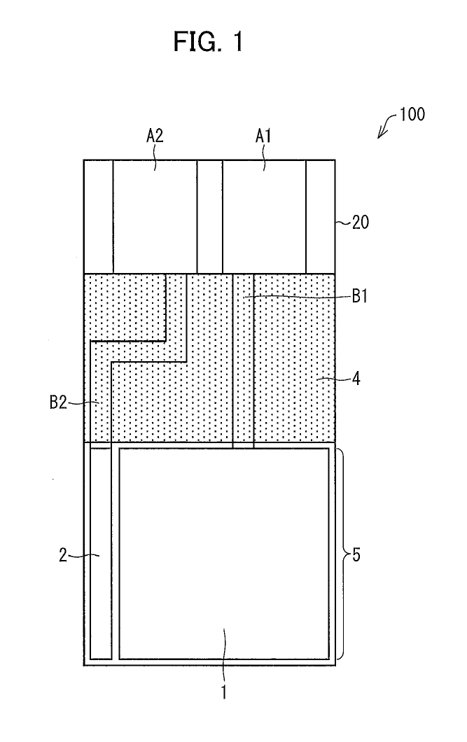

FIG. 1 is a top view schematically illustrating a configuration of a biosensor 100 according to one embodiment (Embodiment 1) of the present invention. As illustrated in FIG. 1, the biosensor 100 includes a working electrode 1, a counter electrode 2, an insulating film 4, a connection pads A1 and A2, lead electrode sections B1 and B2, a reaction section 5, and a substrate 20.

As illustrated in FIG. 1, the biosensor 100 is configured such that the connection pads A1 and A2 are provided on one edge section of the substrate 20, and the working electrode 1 and the counter electrode 2 are provided in juxtaposition on another edge section of the substrate 20, which is opposite to the one edge section. The lead electrode section B1 connects the working electrode and the connection pad A1. The lead electrode section B2 connects the counter electrode 2 and the connection pad A2. Further, the insulating film 4 covers the lead electrodes B1 and B2 so as to prevent the lead electrodes B1 and B2 from being in touch with a sample liquid. Details in the configuration will be discuses later.

The working electrode 1 is an electrode for detecting, by an electrochemical reaction (oxidation or reduction), a product, which is an electrochemical active material produced in the sample liquid. The working electrode 1 may be made from an electrically conductive material such as a metal, carbon, graphite, for example.

The counter electrode 2 is an electrode for flowing a current flow caused by the working electrode 1. The counter electrode 2 may be made from the same electrically conductive material as the working electrode 1 or an electrically conductive material different from the electrically conductive material of the working electrode 1.

The insulating film 4 is a film that is electrically insulating, and is formed to prevent the lead electrode sections B1 and B2 from being in touch with the sample liquid. The insulating film 4 may be made from an electrically insulating material such as polyimide, for example.

The connection pads A1 and A2 are used to connect the electrochemical detecting biosensor 100 to an electrochemical measuring device (for example, potentiostate or the like). The connection pads A1 and A2 are provided to connect the working electrode 1 and the counter electrode 2 with the electrochemical measuring device. The connection pads A1 and A2 may be made from the electrically conductive material from which the working electrode 1 and/or the counter electrode 2 is made. The connection pads A1 and A2 may be made from an electrically conductive material different from the electrically conductive material from which the working electrode 1 and/or the counter electrode 2 is made.

The lead electrode section B1 and B2 are provided to connect the working electrode 1 and the counter electrode 2 with the connection pads A 1 and A2, respectively. The lead electrode sections B1 and B2 are not particularly limited in size (dimension) and may have any size as selected appropriately. The lead electrode sections B1 and B2 may be made from the electrically conductive material from which the working electrode 1 and/or the counter electrode 2 is made.

The substrate 20 is a plate-like or film like part configured to support an electronic unit or the like on its surface so as to realize a function of some sort. For example, the substrate 20 may be made from an electrically insulating material such as glass, quartz, ceramics, plastic, or the like, for example.

The reaction section 5 is a region for holding the sample liquid (that is, a region in touch with the sample liquid) during the electrochemical measurement. The reaction section 5 is configured such that the electrode system including the working electrode 1 and the counter electrode 2 is therein. In the reaction section 5, such a reaction takes place that the target material is directly or gradually reacted with a reaction material or a bonding material immobilized on the working electrode 1 so as to produce the product that is electrochemically active. The biosensor 100 detects the reaction in the reaction section 5 electrochemically by means of the electrode system (working electrode 1 and the counter electrode 2). the reaction material and the bonding material will be discussed later.

The biosensor 100 may be produced as below, for example. By patterning on the substrate 20, the working electrode 1, the counter electrode 2, the connection pads A1 and A2, and the lead electrode sections B1 and B2 are respectively formed. The working electrode 1 may be formed on the substrate 20 by, for example, sputtering, vapor deposition, printing, or the like, followed by patterning. The counter electrode 2, the connection pads A1 and A2, and the lead electrodes B1 and B2 may be formed by a similar manner. Moreover, the biosensor 100 may be mass-produced by dicing a substrate on which sets of the components of the biosensor 100 are provided by patterning.

Next, the insulating film 4 is formed to completely cover the lead electrode sections B1 and B2, in order to prevent the lead electrode sections B1 and B2 from contacting with the sample liquid and from thereby causing a false function of the biosensor 100. This makes it possible to cause the sample liquid to be in touch with the reaction section 5 without being in touch with the other electrically conductive portions of the detecting system of the biosensor 100. The formation of the insulating film 4 over the lead electrode sections B1 and B2 on the surface of the substrate 20 may be carried out by, for example, photolithography, screen printing, or the like.

The formation of the insulating film 4 defines the reaction section 5 with which the sample liquid is to be in touch. The insulating film 4 is formed to have such a size that defines a size (bottom surface) of the reaction section 5 so that the area ratio of the working electrode 1 to the bottom area of the reaction section 5 is 0.7 or greater. By this, it is possible to adjust the size of the reaction section 5 to such a size that the area ratio of the working electrode 1 to the bottom area of the reaction section 5 is 0.7 or greater.

In the present embodiment, an outer border of the reaction section 5 is defined by the insulating film 4. It should be noted by the present invention is not limited to this configuration, and the reaction section 5 may be defined by various ways as described below.

The counter electrode 2 may have an size within a space remained in the reaction section 5 occupied by the working electrode 1. As described above, the counter electrode 2 is an electrode for flowing a current flow caused by the working electrode 1. If the counter electrode 2 is too small relatively to the working electrode 1, it becomes difficult to flow the current, thereby making it difficult to perform the electrochemical measurement accurately. Therefore, it is desirable that the counter electrode 2 has an enough size to cause the current flow between the working electrode 1 and the counter electrode 2.

The connection pads A1 and A2 may be positioned in consideration of where the connection pads A1 and A2 connect the biosensor 100 with the electrochemical measuring device. The connection pads A1 and A2 may be provided at any positions that allow the connection pads A1 and A2 to connect the biosensor 100 with the electrochemical measuring device. Moreover, the connection pads A1 and A2 may have an enough size to be sufficiently connected with connection pads of the electrochemical measuring device.

On the working electrode 1, the reaction material reactive with the target material so as to produce the product, or the bonding material bondable with the target material is immobilized.

The reaction material is a material that reacts with the target material directly to produce the product. The bonding material is a material that reacts with the target material but needs a further reaction to product the product after the reaction with the target material.

In consideration of which target material to be detected, the reaction material or the bonding material can be selected from the group consisting of bio materials such as enzymes, antibodies, peptides, DNAs, oligonucleotides, lectins, receptors, sugars, and the like. For example, in case of detecting a sugar in the sample liquid, an enzyme such as glucose oxidase or the like is selected as the reaction material.

In case where the target material in the sample liquid is detected by immunoassay, an antibody, a peptide, or the like material specifically bondable with the target material is selected as the reaction material or the bonding material. It is preferable that the reaction material or the bonding material is immobilized over a surface of the working electrode 1 wholly.

It is not necessary that the reaction material or the bonding material be immobilized on the surface of the working electrode 1 so densely that the reaction material or the bonding material wholly covers the surface without space. The reaction material or the bonding material may be immobilized on the surface not so densely that the reaction material or the bonding material discretely covers the surface with spaces, provided that the reaction material or the bonding material thus immobilized occupies the surface of the working electrode 1 so that an area ratio of (i) an area occupied with the immobilized reaction material or bonding material on the working electrode 1 to (ii) the bottom surface of the reaction section 5 is 0.7 or greater.

The reaction material or the bonding material may be immobilized on the working electrode 1 by a well-known method, for example, (i) physical adsorption, (ii) a covalent bonding between the reaction material and a functional group provided to the surface of the working electrode 1, (iii) capturing of a protein by a macro molecule having a 3-dimensional net-like structure. If the reaction material or the bonding material is immobilized discretely, a spotter or the like may be used.

The biosensor 100 according to the present embodiment is so configured that the working electrode 1 and the counter electrode 2 are integrally provided on the substrate 20. This configuration provides such an advantage that a small amount of the sample liquid is required to perform the detection.

Note that the working electrode 1, the counter electrode 2, the insulating film 4, the connection pads A1 and A2, the lead electrode sections B1 and B2, the reaction section 5, and the substrate 20 are not particularly limited in terms of shapes and may have shapes different from those exemplified in FIG. 1. For example, the working electrode 1, the counter electrode 2, the insulating film 4, the connection pads A1 and A2, the lead electrode sections B1 and B2, the reaction section 5, and the substrate 20 may be quadrangular, circler, elliptical, or in any other shapes.

[Analysis Method Using Biosensor]

The analysis method using the biosensor comprises: introducing to the reaction section 5 the sample liquid containing the target material; and measuring the electrochemically active material produced as a result of the reaction between the reaction material and the target material or produced as a result of bonding of the bonding material and the target material.

With the configuration of the biosensor 100, the introduction of the sample liquid containing the target material to the reaction section 5 causes the target material to react with the reaction material immobilized on the working electrode 1 so as to produce the product, or causes the target material to bond with the bonding material immobilized on the working electrode. The electrochemical measurement performed after the reaction or bonding detects an ampere value from which the concentration of the target material in the sample liquid can be determined.

To begin with, connection pads A1 and A2 of the electrochemical detecting biosensor are connected with an electrochemical measuring device (for example, potentiostate). The connecting the connection pads A1, and A2 to the electrochemical detecting biosensor may be carried out by, for example, using codes having an alligator clip on either end so that an alligator clip on one end of the codes clips the connection pad A1 and A2 and an alligator clip on another end of the codes clips a terminal of the electrochemical measuring device (for example, potentiostate). However, how to connect the connection pads A1 and A2 to the electrochemical detecting biosensor is not limited to this.

In the following, one example of the analysis method using the biosensor 100 is described below, which is a method for measuring a sugar (glucose) in the sample liquid. It should be noted that the present embodiment is not limited to the example and is applicable to measurement of other kinds of target materials.

In the case of measuring the sugar in the sample liquid, the biosensor 100 is configured such that an enzyme (glucose oxidase) is immobilized on the working electrode 1 as the reaction material. The reaction material may be immobilized on the working electrode 1 by a well-known method such as physical adsorption, covalent bonding between the functional group provided on the surface of the working electrode 1 and the reaction material, and capturing of the protein by using a macro molecule having a 3-dimensional net-like structure, as described above. Moreover, where to immobilize the reaction material on the working electrode 1 is not particularly limited, but it is preferable that the working electrode 1 is immobilized on the surface of the working electrode wholly.

Next, the sample liquid containing glucose is introduced into the reaction section 5. More specifically, the sample liquid is dropped into the reaction section 5 of the biosensor 100. Glucose and the enzyme immobilized on the working electrode 1 reacts with each other, so as to produced the product (hydrogen peroxide). By applying a voltage between the working electrode 1 and the counter electrode 2, a current whose ampere value is varied according to glucose content in the sample liquid flows. By detecting the ampere value of the current, the concentration of glucose in the sample liquid can be measured.

The sample liquid may contain a mediator as a medium for electron movement. The mediator may be such a system as potassium ferrocyanide/potassium ferricyanide, benzoquinone/hydroquinone, ferricinium/ferrocene, or the like. In case of the system for such glucose measurement, a current generated by electron movement via the mediator as a result of the reaction between the enzyme and glucose is measured as a signal. In this way, the concentration of the target material in the sample liquid can be determine from the ampere value thus detected.

Next, another example of the analysis method using the biosensor 100 is described below, which is a method for measuring a minute material in the sample liquid by immunoassay.

In the case of measuring a minute material in the sample liquid by immunoassay, the biosensor 100 is configured such that an antibody or a peptide capable of specifically capturing the target material is immobilized on the working electrode 1 as the bonding material (hereinafter, an analysis method in which an antibody is immobilized is exemplified below, but an analysis method in which a peptide is immobilized is similar to the analysis method exemplified below). The immobilization may be carried out in a manner similar to that of immobilizing the enzyme. Moreover, it is preferable that the bonding material is immobilized over the surface of the working electrode wholly. Moreover, the surface of the working electrode 1 may be subjected to such a treatment before dropping the sample liquid thereto that the surface of the working electrode 1 is treated with an albumin aqueous solution so as to form a anti-unspecific adsorption film on the surface, and is washed with a buffer solution after the formation of the anti-unspecific adsorption film. This treatment prevents non-specific adsorption of the target material to the surface of the working electrode 1.

By dropping the sample liquid containing the target material to the reaction section 5, an antigen-antibody reaction proceeds. Next, the reaction section 5 is washed with a buffer solution, and then a liquid containing an enzyme labeled antibody serving as a second bonding material is dropped to the reaction section 5 for further reaction. By this, a sandwich complex of an antibody-target material-enzyme-labeled antibody is formed on the surface of the working electrode 1. Then, the functional section 5 is washed with a buffer liquid. After that, a sample liquid containing a substrate with which the enzyme reacts is dropped to the reaction section 5. By this, an enzyme-substrate reaction takes place in the sandwich complex formed on the surface of the working electrode 1, thereby producing a product having an electro chemical activity. Consequently, a current varied according to a target material content is flowed when a voltage is applied on the working electrode 1. By detecting the ampere value of the current, the concentration of the target material in the sample liquid can be obtained.

Furthermore, the sample liquid containing the substrate may contain a mediator as a medium for electron movement, as in the case of the other sample liquids described above.

### Embodiment 2

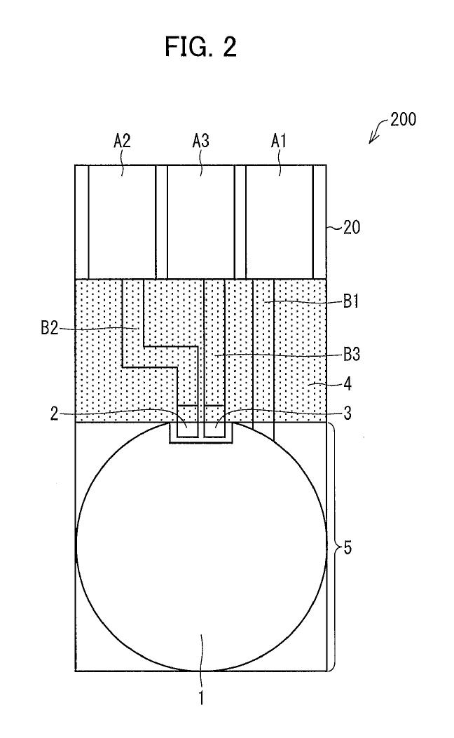

FIG. 2 is a top view schematically illustrating a configuration of a biosensor 200 according to one embodiment (Embodiment 2) of the present invention.

For the sake of easy explanation, like members having like functions illustrated in drawings referred in the explanation in Embodiment 1 are labeled with like reference numerals, and their explanation is not repeated here. Further, analysis methods using the biosensor in the present embodiment are similar to those described above, and their explanation is not repeated here, too.

As illustrated in FIG. 2, the biosensor 200 includes a working electrode 1, a counter electrode 2, a reference electrode 3, an insulating film 4, connection pads A1, A2, and A3, lead electrodes B1, B2, and B3, a reaction section 5, and a substrate 20.

The biosensor 200 is configured such that the connection pads A1, A2, and A3 are provided on one edge section of the substrate 20, and the working electrode 1, the counter electrode 2, and the reference electrode 3 are provided on another edge section of the substrate 20, which is opposite to the one edge section. The lead electrode sections B1, B2, and B3 are configured to connect the working electrode 1 with the connection pad A1, the counter electrode 2 with the connection pad A2, and the reference electrode 3 with the connection pad A3, respectively. Further, the insulating film 4 is formed to cover the lead electrode sections B1, B2, and B3, so as to prevent the lead electrode sections B1, B2, and B3 from being in touch with the sample liquid. This makes it possible to cause the sample liquid to be in touch with the reaction section 5 of FIG. 2 without being in touch with the other electrically conductive portions of the detecting system of the biosensor 200.

As long as the biosensor 200 has the configuration as above and meets the requirement that the area ratio of the working electrode 1 to the reaction section 5 is 0.7 or greater, the members of the biosensor 200 may have any sizes and shapes. Embodiment 2 is different from Embodiment 1 in that Embodiment 2 includes the reference electrode 3.

The reference electrode 3 is an electrode for providing a stable voltage on the working electrode 1. In the biosensor illustrated in FIG. 2, the reference electrode 3 is formed as if the working electrode 1 is inlaid with the reference electrode 3. Where to form the reference electrode 3 is not limited to this position. Considering that a solution resistance would cause an IR drop in the reference electrode 3, it is preferable that the reference electrode 3 is provided as close to the working electrode 1 as possible. As to the size (dimension) thereof, the reference electrode 3 is not particularly limited, but it is preferable that the reference electrode 3 is small in order to form the working electrode 1 with the area ratio of 0.7 or greater with respect to the reaction section 5.

The reference electrode 3 is made from an electrically conductive material, which is preferably such a material that has a stable potential when a current flows therethrough. For example, a silver-silver chloride electrode is one typical example of the reference electrode.

The reference electrode 3 may be formed on the surface of the substrate 20 by, for example, sputtering, vapor deposition, printing, or the like method.

The reference electrode 3 makes it possible to provide a stable voltage on the working electrode 1, thereby enabling more accurate detection.

Moreover, it is preferable that the working electrode 1 is located by being centered in a central portion of a bottom surface of the reaction section 5.

With this configuration, the reaction section 5 can have such a concentration gradient of a diffusion layer of the sample liquid being subject to the electrochemical measurement that the concentration gradient is substantially evenly spread radially about the center of the working electrode 1. This makes it possible to perform the detection in a shorter reaction time, because the electrochemical reaction takes place evenly. The central portion is a region around a center of the reaction section 5 and shares about ⅓ of the total area of the reaction section 5. This configuration only requires that the center of the working electrode 1 be located within the central portion of the bottom surface of the reaction section 5, and is not limited to the geography illustrated in FIG. 2.

Further, it is preferable that a bottom surface of the working electrode 1 is homothetic to the bottom surface of the reaction section 5 in shape.

With this configuration, the reaction section 5 can have such a concentration gradient of a diffusion layer of the sample liquid being subject to the electrochemical measurement that the concentration gradient is substantially evenly spread radially about the center of the working electrode 1. This makes it possible to perform the detection in a shorter reaction time, because the electrochemical reaction takes place evenly. This configuration only requires that the bottom surface of the working electrode 1 and the bottom surface of the reaction section be homothetic in shape, and is not limited to the one illustrated in FIG. 2.

### Embodiment 3

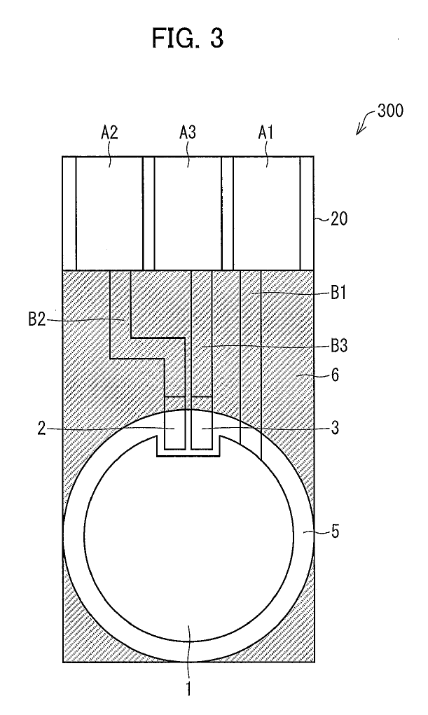

FIG. 3 is a top view schematically illustrating a configuration of a biosensor 300 according to one embodiment (Embodiment 3) of the present invention.

The biosensor 300 as illustrated in FIG. 3 is different from the biosensor 200 of FIG. 2 in that, instead of the insulating film 4, a hydrophobic film (hydrophobic section) 6 is provided to cover the biosensor 300 other than connection pads A1, A2, and A3 and the reaction section 5. Therefore, the reaction section 5 is defined by the hydrophobic film 6. Except for this feature, Embodiment 3 is similar to Embodiment 2.

The hydrophobic film 6 defines the reaction section 5, so that the sample liquid dropped in the reaction 5 is prevented from spreading out of the reaction section 5 by the hydrophobic film 6. In the biosensor 300, the hydrophobic film 6 is configured to cover the potion of the biosensor 300 around and except the reaction section 5.

The hydrophobic film 6 is made from a material having a hydrophobic surface and an electrically insulating property. The hydrophobic film 6 may be formed by, for example, (i) hydrophobic polymer coating, (ii) chemically modification with a toluene solution of octadodecyl trichloro silane, or (iii) the other appropriate method.

The hydrophobic film 6 defines the reaction section 5, thereby restricting the sample liquid to be spreadable only within the reaction section 5. This makes it possible to perform the detection with the sample liquid of an amount just required for the detection.

### Embodiment 4

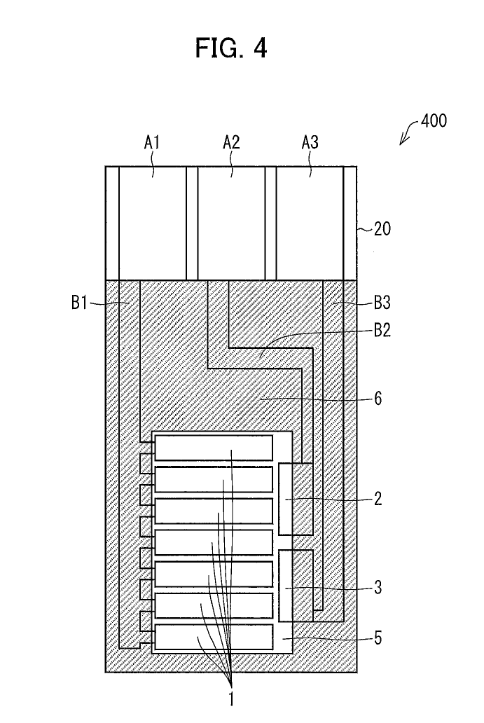

FIG. 4 is a top view schematically illustrating a configuration of a biosensor 400 according to one embodiment (Embodiment 4) of the present invention.

The electrochemical detecting biosensor 400 as illustrated in FIG. 4 is configured such that it includes a plurality of working electrodes 1 and a lead electrode section B1, and each of the working electrodes 1 is connected with a connection pad A1 via the lead electrode section B1. Except this, Embodiment 4 is similar to Embodiment 3.

A total area summing each area of the working electrodes 1 is in a ratio of 0.7 or greater to a bottom surface of the reaction section 5.

The plurality of working electrodes 1, each of which is small in area, provide an effect of “micro electrodes” to amplify an ampere value, thereby making it possible to perform highly sensitive detection.

### Embodiment 5

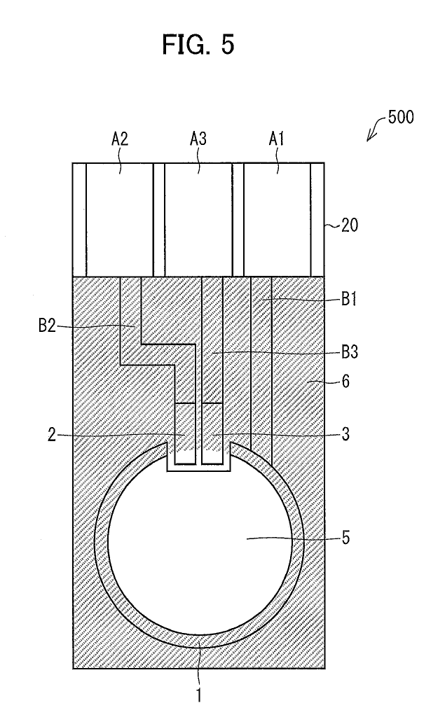

FIG. 5 is a top view schematically illustrating a configuration of a biosensor 500 according to one embodiment (Embodiment 5) of the present invention.

The biosensor 500 as illustrated in FIG. 5 is similar to the biosensor 300 of Embodiment 3, except that a working electrode 1 is larger in size than a reaction section 5 in the biosensor 500. The hydrophobic film 6 defines an effective area of the working electrode 1 within the reaction section 5. In the biosensor 500, the effective area of the working electrode 1 has the area ratio of 0.7 or greater with respect to the reaction section 5.

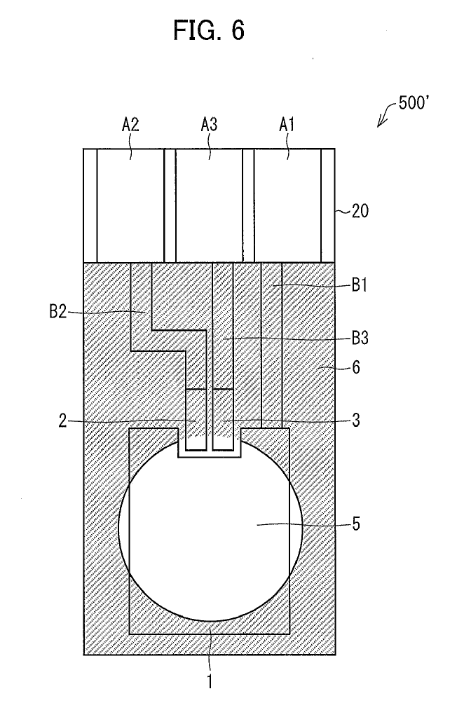

All peripheries of the working electrode 1 may be extended beyond the reaction section 5 as in the biosensor 500 illustrated in FIG. 5, or one or some peripheries of the working electrode 1 may be extended beyond the reaction section 5 as in a biosensor 500′ illustrated in FIG. 6.

### Embodiment 6

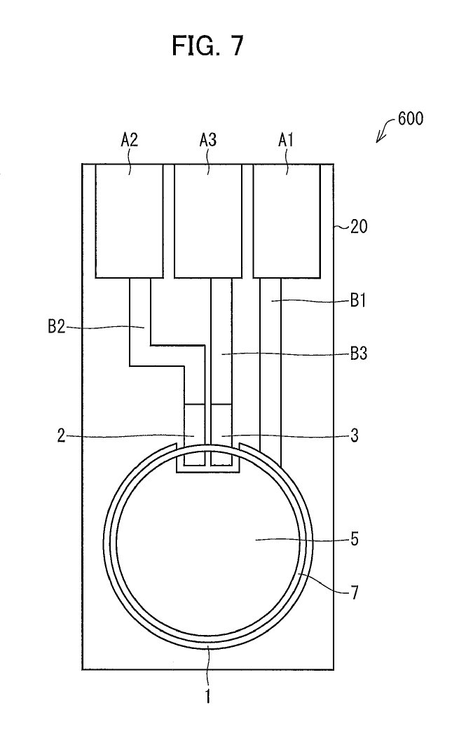

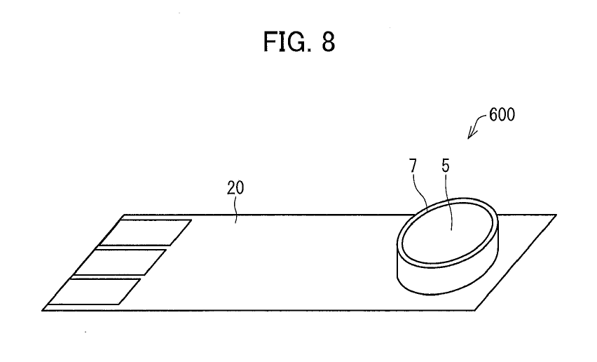

FIG. 7 is a top view schematically illustrating a configuration of a biosensor 600 according to one embodiment (Embodiment 6) of the present invention. FIG. 8 is a perspective view schematically illustrating the configuration of the biosensor 600 according to Embodiment 6. It should be noted that the detailed structure such as working electrode 1 etc. is omitted from the illustration in FIG. 8.

The biosensor 600 is similar to the biosensor 500 of Embodiment 5, except that a reaction section 5 is defined by a wall 7 surrounding the reaction section 5, and that the hydrophobic film 6 is not provided to cover the biosensor 600.

The wall 7 is configured to define the reaction section 5, so that the sample liquid dropped in the reaction section 5 is prevented from spreading out of the reaction section 5. In the biosensor 600, the wall 7 has a ring-like shape to surround, in a plan view, the reaction section 5 having a circle shape. It should be noted that the wall 7 is not limited to this shape, and may have any shape in accordance with the shape of reaction section 5. Moreover, in terms of height, the wall 7 is only required to have a height enough to prevent the sample liquid from spreading over the wall 7. The wall 7 may be made from glass, quartz, ceramics, plastic, or the like. If the wall 7 is made from polydimethyl siloxane (PDMS), process and mass production of the biosensor can be easier.

The wall 7 may be formed by, for example, mechanical processing, chemical processing (such as etching), or the other method. How to form the wall 7 is not particularly limited. Moreover, the wall 7 may be formed by molding an light- or heat curable resin in a mold patterned according to the components of the biosensor. Furthermore, the wall 7 may be formed by hot embossment of a material such as polyolefin resin, polymethacrylic resin, polycarbonate resin, or the like, by using a mold patterned according to the components of the biosensor.

The wall 7 thus formed is attached to the substrate 20, thereby defining the reaction section 5.

The wall 7 can surely prevent the sample liquid from spreading out of the reaction section 5.

### Embodiment 7

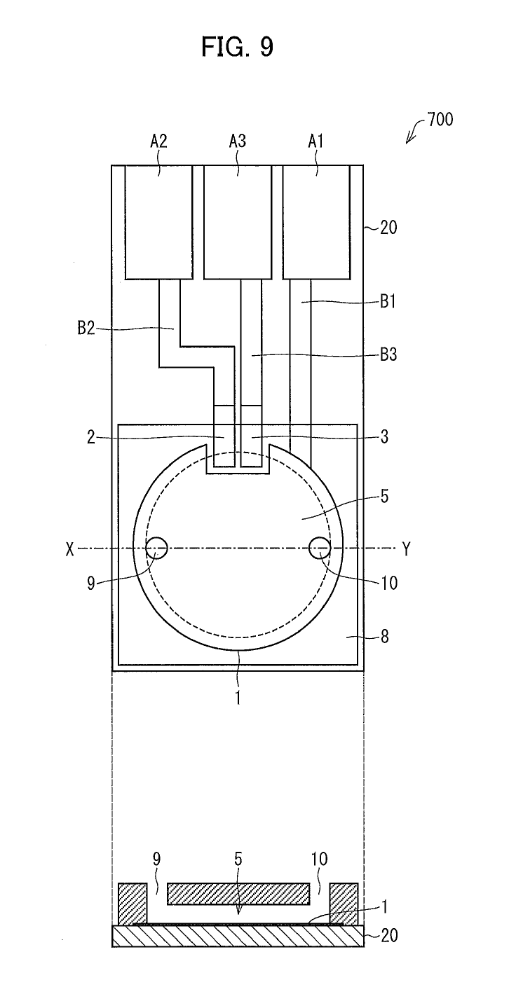

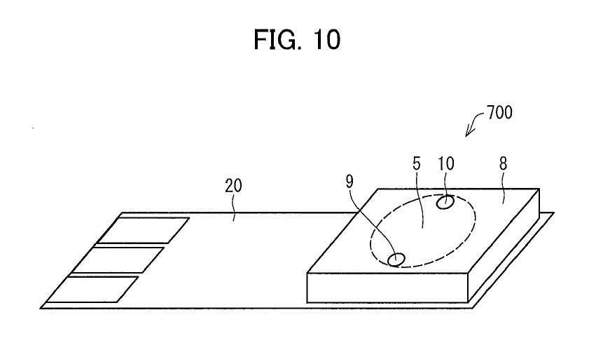

FIG. 9 is a view schematically illustrating a top view and a cross sectional view of a configuration of a biosensor 700 according to one embodiment (Embodiment 7) of the present invention. FIG. 10 is a perspective view schematically illustrating the configuration of the biosensor 700 according to Embodiment 7. It should be noted that the detailed structure such as working electrode 1 etc. is omitted from the illustration in FIG. 10.

A reaction chamber 8 includes a wall and a ceiling portion surrounding a reaction section 5, and thereby defines the reaction 5 3-dimensionally. Further, the reaction chamber 8 has an inlet section 9 for introducing a liquid into the reaction chamber 8, and an outlet section 10 for discharging the liquid out of the reaction chamber 8.

The biosensor 700 is similar to the biosensor 200 of Embodiment 2, except that the reaction chamber 8 having the inlet section 9 and the output section 10 is provided on the substrate 20 and the insulating film 4 for covering is not provided in the biosensor 700.

The reaction chamber 8, which is illustrated as a 3-dimensional shape having a circular column-like shape, is not limited to the shape as illustrated and may have any shape in accordance with the shape of the reaction section 5. The reaction chamber 8 may be made from glass, quartz, ceramics, plastics, or the like. If the reaction chamber 8 is made from polydimethyl siloxane (PDMS), process and mass production of the biosensor can be easier.

The reaction chamber 8 may be formed by a method similar to the method forming the wall 7. How to form the reaction chamber 8 is not particularly limited.

The reaction chamber 8 defining the reaction section 5 3-dimensionally can surely prevent the sample liquid from spreading out of the reaction section 5.

The reaction chamber 8 is configured to define the reaction section 5 3-dimensionally.

The inlet section 9 is configured to introduce the sample liquid or the like into the reaction section 5.

The outlet section 10 is configured to discharge the sample liquid or the like out of the reaction section 5 in which the sample liquid or the like is introduced. Furthermore, the output section also can serve as an exhaust outlet 10 in introducing the sample liquid or the like into the reaction chamber 8.

The biosensor 700 is configured such that the inlet section 9 and the outlet section 10 are formed as opening having a circular shape and being opened in communication with the reaction section 5. However, the inlet section 9 and the outlet section 10 may be any shape, provided that the inlet section 9 and the outlet section 10 allow liquid transfer therethrough.

The discharge of the liquid may be carried out via the inlet section 9. In this case, the outlet section 10 is used as an exhaust outlet.

In the following, an analysis method using the electrochemical detecting biosensor 700 described in Embodiment 7. The analysis method is similar to those in Embodiments 1 to 6, except that the sample liquid, the buffer liquid, or the like is introduced to the reaction section 5 via the inlet section 9 and is discharged out of the reaction section 5 via the outlet section 10 in Embodiment 7, instead of dropping the sample liquid etc. in the reaction section 5 in Embodiments 1 to 6.

The invention being thus described, it will be obvious that the same way may be varied in many ways. Such variations are not to be regarded as a departure from the spirit and scope of the invention, and all such modifications as would be obvious to one skilled in the art are intended to be included within the scope of the following claims.

### SUMMARY

As described above, a biosensor according to the present invention is a biosensor comprising a working electrode or working electrodes on which a reaction material or a bonding material is immobilized, where the reaction material is reactive with a target material so as to produce a product, and the bonding material is bondable with a target material; a counter electrode; and a reaction section for holding a sample liquid containing the target material, the working electrode and the counter electrode being provided on a bottom surface of the reaction section, and the working electrode occupying the bottom surface of the reaction section by a ratio of 0.7 or greater.

Compared with the conventional biosensor, this configuration makes it possible to reduce a difference between an actual initial reaction rate and a calculated initial reaction rate in an reaction initial stage of a reaction for producing the product, the calculated initial reaction rate being obtained from a gradient of a straight line connecting an origin and a product amount at a given time. Therefore, this configuration makes it possible to perform accurate detection with a short reaction time without requiring to wait for the reaction to saturate.

This is explained herein for further details. The conventional biosensors (Patent Literatures 1 to 4) is configured such that the area ratio of the working electrode (measuring electrode) to an area to be in touch with the sample liquid or measuring-target liquid on a substrate is small. This is because these conventional biosensors are so configured that the counter electrode for flowing the current caused by the working electrode has an area ratio substantially equal to or greater than that of the working electrode in order to avoid difficulty in flowing the current through the counter electrode. Thus, the conventional biosensors are so configured that the working electrode occupies, by an area ratio of 0.5 or less, the area to be in touch with the liquid.

Here, in general, the product produced on the working electrode gradually move away from the working electrode by diffusion. The electrochemical detection is capable of detecting only the product present in the vicinity of the working electrode. If the area ratio of the function electrode to the bottom area of the reaction section is 0.5 as in the conventional biosensors, a portion not the working electrode is large in the bottom area of the reaction section. This follows that an amount of the product moving out of detectable range due to the diffusion is large. Consequently, the reaction time and the product amount (product amount on the working electrode) has low linearity in the reaction initial stage.

On the other hand, in the biosensor in which the area ratio of the working electrode to the bottom surface of the reaction section as described above is 0.7 or greater, the portion not the working electrode is small in the bottom area of the reaction section. This follows that an amount of the product moving out of detectable range due to the diffusion is small. Consequently, the reaction time and the product amount (product amount on the working electrode) has high linearity even in the reaction initial stage, that is, the reaction time and the product amount has a more linear relationship therebetween. Because of this, this configuration makes it possible to reduce a difference between an actual initial reaction rate and a calculated initial reaction rate in an reaction initial stage of a reaction for producing the product, the calculated initial reaction rate being obtained from a gradient of a straight line connecting an origin and a product amount at a given time, whereby this configuration makes it possible to perform accurate detection even with a short reaction time.

It is preferable that the biosensor according to the present invention comprises a plurality of the working electrodes.

With this configuration, the plurality of working electrodes, each of which is small in area, provide an effect of “micro electrodes” to amplify an ampere value, thereby making it possible to perform highly sensitive detection.

It is preferable that the biosensor according to the present invention further comprises a reference electrode.

With this configuration, it becomes possible to provide a stable voltage on the working electrode.

It is preferable that the biosensor according to the present invention comprises a hydrophobic portion having a hydrophobic property and surrounding the reaction section.

With this configuration in which the reaction section is surrounded by the hydrophobic portion, the liquid in the reaction section is prevented from spreading out of the reaction section. This makes it possible to perform the detection with a drop of the sample liquid, for example.

It is preferable that the biosensor according to the present invention comprises a wall surrounding the reaction section.

With this configuration, the wall surrounding the detection electrode prevent the liquid from spreading out of the reaction section where the working electrode (detecting electrode) is present. This makes it possible to successfully perform the detection with a minute amount of sample liquid.

It is preferable that the biosensor according to the present invention comprises a reaction chamber, in which the reaction section is contained, the reaction chamber having an inlet for introducing the sample liquid into the reaction chamber via the inlet, and an outlet for discharging the sample liquid out of the reaction chamber via the outlet.

With this configuration, the reaction section can be contained in the reaction chamber, thereby making possible to perform the detection with a more minute amount of sample liquid and a shorter measuring time by more simple operation. This makes it possible to measure a sample accurately and efficiently in a shorter time.

The biosensor according to the present invention is preferably configured such that the working electrode is located by being centered in a central section of the bottom surface of the reaction section.

With this configuration, the reaction section can have such a concentration gradient of a diffusion layer of the sample liquid being subject to the electrochemical measurement that the concentration gradient is substantially evenly spread radially about the center of the working electrode. This makes it possible to perform the detection in a shorter reaction time, because the electrochemical reaction takes place evenly. The central portion is a region around a center of the reaction section and shares about ⅓ of the total area of the reaction section.

The biosensor according to the present invention is preferably configured such that the working electrode has a bottom surface having a shape homothetic to a shape of the bottom surface of the reaction section.

With this configuration, the reaction section can have such a concentration gradient of a diffusion layer of the sample liquid being subject to the electrochemical measurement that the concentration gradient is substantially evenly spread radially about the center of the working electrode. This makes it possible to perform the detection in a shorter reaction time, because the electrochemical reaction takes place evenly.

The biosensor according to the present invention is preferably configured such that the reaction material is reactive specifically with the target material. Furthermore, the biosensor according to the present invention is preferably configured such that the reaction material is an enzyme for catalyzing a reaction of the target material.

This configuration makes it possible to detect, as the target material, a substrate reactive with an enzyme.

The biosensor according to the present invention is preferably configured such that the bonding material is bondable specifically with the target material. Furthermore, the biosensor according to the present invention is preferably configured such that the bonding material is an antibody for the target material or a peptide bondable specifically with the target material.

With this configuration, immunoassay becomes possible by further reacting with a second bonding material reactive with the target material. The second bonding material is a material bondable with the target material and reactive with a substrate so as to produce a product. One example of the second bonding material is an enzyme-labeled antibody.

An analysis method according to the present invention is an analysis method using the aforementioned biosensor, comprising: introducing, to the reaction section, the sample liquid containing the target material; and measuring an ampere value of a current caused by voltage application between the working electrode and the counter electrode, the ampere value being varied according to an amount of the target material reacted or bonded.

With this configuration, the step of introducing introduces the sample liquid containing the target material to the reaction section. This causes reaction between the reaction material immobilized on the working electrode and the target material or bonding between the bonding material on the working electrode and the target material. By applying a voltage between the working electrode and the counter electrode, an ampere value being varied according to an amount of the target material reacted or bonded can be obtained. The configuration of the present invention provides a high linearity (more liner relationship) between the reaction time and the product amount of the product in the reaction initial stage. Because of this, this configuration makes it possible to reduce a difference between an actual initial reaction rate and a calculated initial reaction rate in an reaction initial stage of a reaction for producing the product, the calculated initial reaction rate being obtained from a gradient of a straight line connecting an origin and a product amount at a given time, whereby this configuration makes it possible to perform accurate detection even with a short reaction time.

### EXAMPLES

### Example 1

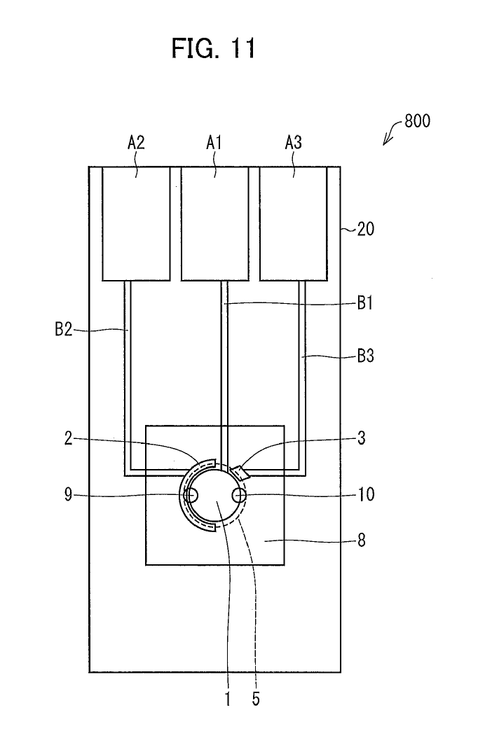

FIG. 11 is a top view schematically illustrating a configuration of a biosensor 800 used in Example 1. Example 1 is explained below, referring to FIG. 11.

On a glass wafer (Corning Incorporated; Eagle XG) of 10 cm×10 cm in size and 0.5 mm in thickness, a working electrode 1, a counter electrode 2, a connection pads A1, A2, and A3, lead electrode sections B1, B2, and B3 were formed by sputtering gold on the glass wafer and then performing photolithography on the sputtered gold. The working electrode 1 was formed to have a circular shape of 2 mm in diameter, and the counter electrode 2 was formed to have a circular arc shape surrounding the working electrode 1.

Next, a silver electrode was formed in the same way by photolithography. Part of the silver electrode was converted into silver chloride chemically, thereby forming a reference electrode 3 made of silver and silver chloride.

In this way, a plurality of electrode substrates (1 cm×2 cm) as illustrated in FIG. 11 were formed the glass wafer. Then, the glass wafer was diced into the individual electrode substrates by using a glass cutter.

A mold for producing a reaction chamber was prepared on a silicon wafer, so as to prepare a reaction chamber having a bottom surface having a circular shape of 2.3 mm in diameter and height of 40 μm. Into the mold, polydimethyl siloxane (PDMS) was poured in, and thermally solidified, thereby preparing the reaction chamber. On both edge sections of the reaction chamber, an opening of 0.5 mm in diameter was formed, thereby preparing an inlet section 9 and an outlet section 10.

On the working electrode 1 of the electrode substrate, a self-assembled monolayer (SAM) of thiol molecules was formed and glucose oxidase was immobilized on the working electrode 1 via the self-assembled monolayer.

The reaction chamber was attached to the substrate to which the glucose oxidase was immobilized, thereby producing the biosensor 800 for use in Example 1.

An area ratio of the working electrode 1 to a bottom surface of the reaction section 5 thus formed by the reaction chamber was 0.76 in Example 1.

### Comparative Example 1

A mold for producing a reaction chamber was prepared on a silicon wafer, so as to prepare a reaction chamber having a bottom surface having a circular shape of 2.8 mm in diameter and height of 40 μm. Into the mold, polydimethyl siloxane (PDMS) was poured in, and thermally solidified, thereby preparing the reaction chamber.

The reaction chamber was attached to a substrate which was prepared in the same way as in Example 1 and to which the glucose oxidase was immobilized, thereby producing a biosensor for use in Comparative Example 1.

An area ratio of the working electrode 1 to a bottom surface of the reaction section 5 thus formed by the reaction chamber was 0.51 in Comparative Example 1.

(Glucose Detection)

By using the biosensors of Example 1 and Comparative Example 1, glucose detection was carried out for glucose solutions of 50 mg/dL, 100 mg/dL, and 250 mg/dL.

The glucose solution was introduced in the reaction section 5, and an ampere value was measured at 10 sec after the introduction. The ampere value thus measured was corrected with a background current, and then plotted against time, thereby obtaining a straight line between the origin and the ampere value thus plotted. From the straight line, an initial reaction rate was obtained.

In case of the Comparative Example 1 with the area ratio of 0.51, the glucose concentration was not proportional to the initial reaction rate. On the contrary, in the case of Example 1 with the area ratio of 0.76, the glucose concentration was proportional to the initial reaction rate, and the initial reaction rate determined from a gradient of the straight line was substantially equal to an actual initial reaction rate.

This confirmed that the use of the biosensor according to the present invention is capable of accurately determining a initial reaction rate of glucose measurement for a glucose solution with an unknown glucose concentration with a short reaction time, and thereby determining the glucose concentration in the glucose solution by comparing the determined initial reaction rate with initial reaction rates of glucose measurement for glucose solutions with known concentrations.

### Example 2

On a working electrode 1 of an electrode substrate prepared in the same way as in Example 1, anti-CRP antibody was immobilized via a self-assembled monolayer (SAM) of thiol molecules formed on the working electrode 1. A reaction chamber identical with the one used in Example 1 was attached to the electrode substrate, thereby preparing a biosensor 800 for use in Example 2.

### Comparative Example 2

On a working electrode 1 of an electrode substrate prepared in the same way as in Example 1, anti-CRP antibody was immobilized via a self-assembled monolayer (SAM) of thiol molecules formed on the working electrode 1 as in Example 2. A reaction chamber identical with the one used in Comparative Example 1 was attached to the electrode substrate, thereby preparing a biosensor for use in Comparative Example 2.

(CRP Detection)

By using the biosensors of Examples 2 and Comparative Example 2, CRP detection was performed. To begin with, a casein solution was introduced in the reaction section of the biosensors of Examples 2 and Comparative Example 2, and let stand at room temperature for 30 min, thereby an anti-unspecific adsorption film. After the casein solution was discharged, the reaction section inside was washed with a PBS solution. Then, a CRP solution having a concentration of 0.2 mg/dL, 2 mg/dL, or 10 mg/dL was introduced in the reaction section and let stand at room temperature for 3 min, so as to form, on the working electrode, a complex of (i) the antibody immobilized on the working electrode and (ii) CRP. After the CRP solution was discharged, the reaction section inside was washed. Then, an ALP-labeled anti CRP antibody serving as a second bonding material was introduced in the reaction section and let stand at room temperature for 3 min, thereby forming a sandwich complex of the immobilized antibody-CRP-the ALP-labeled antibody. ALP is an enzyme called alkaline phosphatase. After the ALP-labeled antibody was discharged, the reaction section inside was washed. Then, p-aminophenyl phosphate (pAPP) solution was introduced in the reaction section, an ampere value was measured at 10 sec after the introduction of the pAPP solution. The ampere value thus measured was corrected with a background current, and then plotted against time, thereby obtaining a straight line between the origin and the ampere value thus plotted. From the straight line, an initial reaction rate was obtained.

In case of the Comparative Example 2 with the area ratio of 0.51, the CRP concentration was not proportional to the initial reaction rate. On the contrary, in the case of Example 2 with the area ratio of 0.76, the CRP concentration was proportional to the initial reaction rate, and the initial reaction rate determined from a gradient of the straight line was substantially equal to an actual initial reaction rate.

This confirmed that the use of the biosensor according to the present invention is capable of accurately determining a initial reaction rate of CRP measurement for a CRP solution with an unknown CRP concentration, and thereby determining the CRP concentration in the CRP solution by comparing the determined initial reaction rate with initial reaction rates of CRP measurement for CRP solutions with known concentrations.

It should be noted that the present invention is not limited to CRP, and is also applicable to immunoassay in general.

### INDUSTRIAL APPLICABILITY

The biosensor according to the present invention is capable of determining a concentration of a target material in a sample liquid from a detected ampere value of a current by electrochemical measurement. Thus, the biosensor according to the present invention is applicable to analysis of samples relating to biological objects, environments, medical objects, and foods, etc.

### REFERENCE SIGNS LIST

1: Working electrode
2: Counter Electrode
3: Reference electrode
A1 to A3: Connection Pads
B1 to B3: Lead Electrode Section
4: Insulating Film
5: Reaction Section
6: Hydrophobic Film
7: Wall
8: Reaction Chamber
9: Inlet Section
10: Outlet Section
20: Substrate

1. A biosensor comprising:
a working electrode or working electrodes on which a reaction material or a bonding material is immobilized, where the reaction material is reactive with a target material so as to produce a product, and the bonding material is bondable with a target material;
a counter electrode; and
a reaction section for holding a sample liquid containing the target material,
the working electrode and the counter electrode being provided on a bottom surface of the reaction section, and
the working electrode occupying the bottom surface of the reaction section by a ratio of 0.7 or greater.

2. The biosensor as set forth in claim 1, comprising a plurality of the working electrodes.

3. The biosensor as set forth in claim 1, further comprising a reference electrode.

4. The biosensor as set forth in claim 1, comprising a hydrophobic portion having a hydrophobic property and surrounding the reaction section.

5. The biosensor as set forth in claim 1, comprising a wall surrounding the reaction section.

6. The biosensor as set forth in claim 1, comprising:
a reaction chamber, in which the reaction section is contained,
the reaction chamber having an inlet for introducing the sample liquid into the reaction chamber via the inlet, and an outlet for discharging the sample liquid out of the reaction chamber via the outlet.

7. The biosensor as set forth in claim 1, wherein the working electrode is located by being centered in a central section of the bottom surface of the reaction section.

8. The biosensor as set forth in claim 7, wherein the working electrode has a bottom surface having a shape homothetic to a shape of the bottom surface of the reaction section.

9. The biosensor as set forth in claim 1, wherein the reaction material is reactive specifically with the target material.

10. The biosensor as set forth in claim 9, wherein the reaction material is an enzyme for catalyzing a reaction of the target material.

11. The biosensor as set forth in claim 1, wherein the bonding material is bondable specifically with the target material.

12. The biosensor as set forth in claim 11, wherein the bonding material is an antibody for the target material or a peptide bondable specifically with the target material.

13. An analysis method using a biosensor as set forth in claim 1, comprising:
introducing, to the reaction section, the sample liquid containing the target material; and
measuring an ampere value of a current caused by voltage application between the working electrode and the counter electrode, the ampere value being varied according to an amount of the target material reacted or bonded.
